(19) United States
(12) Patent Application Publication
(10) Pub. No.: US 2002/0161460 A1
(43) Pub. Date: Oct. 31, 2002

(54) Automatic washer disinfector apparatus

(75) Inventor: Toshiaki Noguchi, Tachikawa-shi (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: Olympus Optical Co., Ltd., Tokyo (JP)

(21) Appl. No.: 10/175,204

(22) Filed: Jun. 19, 2002

Related U.S. Application Data
(63) Continuation of application No. PCT/JP00/07117, filed on Oct. 13, 2000.

(51) Int. Cl. G05B 11/01; G05B 19/42; G06F 17/00

(52) U.S. Cl. 700/90; 700/89; 700/24

(57) ABSTRACT

An automatic washer disinfector apparatus performs cleaning/disinfection of an object under program control, a data storage medium which stores therein identification data concerning the object is individually provided on each object to be cleaned/disinfected, and at least one RFID transmission/reception circuit main body which enables data communication with the data storage medium when an arbitrary object is set in a cleaning chamber of the automatic washer disinfector apparatus is provided on the inside, the surface or the outer side of the automatic washer disinfector apparatus. Further, an appropriate program is selected from a plurality of cleaning/disinfection programs based on the identification data of the object obtained by data communication with the data storage medium, and the selected program is activated, thereby cleaning/disinfecting the object.

### CROSS REFERENCE TO RELATED APPLICATIONS

This is a Continuation Application of PCT Application No. PCT/JP00/07117, filed Oct. 13, 2000, which was not published under PCT Article 21(2) in English.

### BACKGROUND OF THE INVENTION

1. Field of the Invention

The present invention relates to an automatic washer disinfector apparatus.

2. Description of the Related Art

The prior art will be described with reference to FIGS. 11A and 11B and FIGS. 12A to 12D. There is an industry that dedicatedly mechanically adjusts (cleaning, cleansing, drying and disinfecting) for reuse medical equipment such as an endoscope 101 (FIG. 12A), a treatment instrument 102 (FIG. 12B), a rigid endoscope 103 (FIG. 12C) or a trocar 104 (FIG. 12D) in an automatic washer disinfector apparatus having internally stored programs or an automatic washer disinfector apparatus in the environment of a hospital or a laboratory. In such an industry, the obligation for performing the validity check of cleaning/disinfection processes and for documentation of the check result further assures an importance.

For complete documentation, various objects which are brought into a cleaning chamber or a disinfection chamber of the automatic washer disinfector apparatus for the purpose of adjustment for reuse must be all recorded, and important data concerning processing such as cleaning must be assuredly stored in a memory based on a simultaneous or immediate data processing protocol.

Such a measure is designed in view of regular handling and adjustment of a medical cleaning object in the washer apparatus.

FIG. 11A shows an overview of an automatic washer disinfector apparatus, and in an automatic washer disinfector apparatus main body 1 are provided a cleaning chamber 2 which accommodates therein medical equipment which is a cleaning/disinfection object and performs cleaning/disinfection, a door 3 used for protection so that medical fluid or the like is not scattered during cleaning, and an operation panel 4 for controlling/manipulating the operation of the automatic washer disinfector apparatus.

Further, there is also provided a barcode reader 5 which can read the above-described cleaning object or its container (for example, a cleaning object holder 7) by using a scanner and store the read information in a data processing device, and a barcode reader control main body 6.

In the above-mentioned automatic cleaning/disinfecting method, there is known a method which confirms a cleaning object to be used and record the cleaning object before attaching a medical apparatus in a cleaning tank so that data of such equipment can be rechecked by the data protocol or again called.

In the case of cleaning/disinfecting the endoscope 101 (FIG. 12A), a data storage medium (barcode in this example) 10′ attached to a universal connector 9 of the endoscope is read by the barcode reader 5, and the endoscope main body is attached to the cleaning chamber 2.

Then, the automatic washer disinfector apparatus main body 1 determines the read data, and activates a processing program suitable for the cleaning/disinfection object, selected from programs which are stored in the automatic washer disinfector apparatus.

Furthermore, in the case of cleaning/disinfecting the treatment instrument 102 (FIG. 12B), the rigid endoscope 103 (FIG. 12C), the trocar 104 (FIG. 12D) and the like, they are accommodated in the holder 7 having a barcode 8 as the data storage medium attached on a side surface thereof, the holder 7 is then accommodated in the cleaning chamber 2 of the automatic washer disinfector apparatus, and a processing program suitable for the cleaning/disinfection object is activated, as in the case of the endoscope.

A similar effect can be obtained when a data storage medium (barcode in this example) 10′ is attached to the treatment instrument 102 (FIG. 12B), the rigid endoscope or the trocar as in the case of attaching to the side surface of the holder 7.

In the above-described prior art, the method using the barcode for identifying a plurality of medical equipments or apparatuses has a drawback in that when the cleaning object identified outside the automatic washer apparatus before cleaning is actually taken in the automatic washer apparatus at the time of cleaning, it is sometimes not cleaned.

### BRIEF SUMMARY OF THE INVENTION

It is an object of the present invention to provide an automatic washer disinfector apparatus which can identify inherent data of a cleaning object, easily enable analysis of an operation protocol for apparatuses by recording data important for a cleaning procedure, and carry out appropriate cleaning/disinfection by optimally selecting cleaning conditions of the cleaning object.

According to the present invention, there is provided an automatic washer disinfector apparatus which performs cleaning/disinfection of an object under program control, wherein a data storage medium which stores therein identification data concerning each object is individually provided on each object to be cleaned/disinfected, one transmission/reception device, which enables data communication with the data storage medium when an arbitrary object is set in a cleaning chamber of the automatic washer disinfector apparatus, is provided on the inside, the surface or the outer side of the automatic washer disinfector apparatus, an appropriate program is selected from a plurality of cleaning/disinfection programs based on the identification data of the object acquired by data communication with the data storage medium, and cleaning/disinfection of the object is carried out by activating this selected program.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIGS. 1A to 1D are views showing examples of provision of an RFID (Radio Frequency Identification) such as a transponder(transmission responder) is provided as a data storage medium on a cleaning/disinfection object;

FIGS. 2A and 2B are views showing external appearances of the RFID (transponder);

FIG. 3 is a conceptual view of the RFID (transponder);

FIGS. 4A, 4B and 4C are views illustrating a first embodiment according to the present invention;

FIG. 5 is a view showing an example that a “tag” is attached to a universal code of an endoscope;

FIG. 6 is a flowchart for illustrating the operation of the first embodiment according to the present invention;

FIG. 7 is a view for illustrating a second embodiment according to the present invention;

FIG. 8 is a view for illustrating the second embodiment of the present invention;

FIGS. 9A and 9B are views for illustrating a third embodiment of the present invention;

FIG. 10 is a view for illustrating a fourth embodiment according to the present invention;

FIGS. 11A and 11B are views showing a structure of a prior art automatic washer disinfector apparatus; and

FIGS. 12A to 12D are views showing examples that a barcode is provided as a data storage medium on the cleaning/disinfection object.

### DETAILED DESCRIPTION OF THE INVENTION

An outline of an embodiment will be first described. In this embodiment, medical equipments or machines such as an endoscope or a set of MIC (minimally invasive cardiac surgery) instruments aligned in a cleaning object wagon or holder fitted in a cleaning chamber of the automatic washer disinfector apparatus having internally stored programs is identified, inherent data of the cleaning object stored in a specific storage device is identified from a result of the identification, and a processing program suitable for a cleaning/disinfection object is activated from selectable processing (adjustment) programs stored in an automatic washer disinfector apparatus.

Therefore, each piece of medical equipment brought into the cleaning chamber of this apparatus is individually provided with one data storage medium which stores therein identification data of that piece of medical equipment. Further, at least one transmission/reception device is incorporated on the inside, the surface or the outer side of the cleaning chamber of the automatic washer disinfector apparatus and can be electromagnetically coupled so data transmission can be performed with these data storage mediums.

Furthermore, the respective cleaning objects aligned in the cleaning chamber in association with a common or a plurality of transmission/reception devices are identified in accordance with the type of cleaning object or the identification data inherent to the cleaning objects or the cleaning program, and the obtained identification data is stored. Subsequently, there is activated one processing program which is induced from the detected identification data and selected with respect to all the loaded cleaning objects.

A description will now be given as to the structure of the automatic washer disinfector apparatus according to this embodiment. In this embodiment, means capable of storing data as well as reading data is provided between the automatic washer disinfector apparatus and the cleaning/disinfection object in a non-contact manner, thereby solving the problems in the prior art.

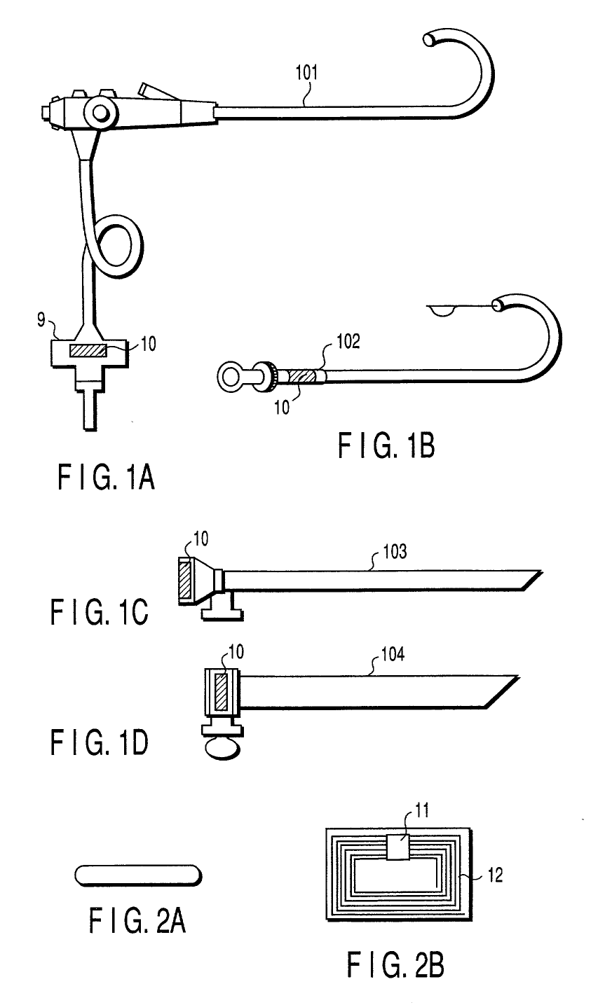

FIGS. 1A to 1D show examples of provision of an RFID (Radio Frequency Identification), such as transponder, on the cleaning/disinfection object as the data storage medium 10 instead of the conventional barcode 8.

An RFID (transponder) is provided as the data storage medium (data memory) 10 on a part of a universal connector 9 of a microscope 101 in FIG. 1A, an RFID (transponder) is provided as the data storage medium (data memory) 10 on the treatment instrument 102 in FIG. 1B, the same is provided on the rigid endoscope 103 in FIG. 1C, and the same is provided on the trocar 104 in FIG. 1D.

The characteristic of the RFID (transponder) lies in that data can be read and written in a non-contact manner, which differs from a barcode, and there are few restrictions in installation conditions of the devices (the RFID transmission/response circuit and the data storage medium) during reading. On the contrary, in the case of a barcode, restrictions of distance or angle between the reader and the barcode label are rigid, and the reading accuracy is low.

FIGS. 2A and 2B show external appearances of the RFID (transponder). FIG. 2A shows the RFID which accommodates all of the transponder in a glass tube and is superior in the environment-resistant property. FIG. 2B shows a transponder which is of a label type, and an RFID chip 11 and an antenna 12 are constituted on a flat surface. This is characterized in the affordable price and convenience. These types can be separately used depending on purpose.

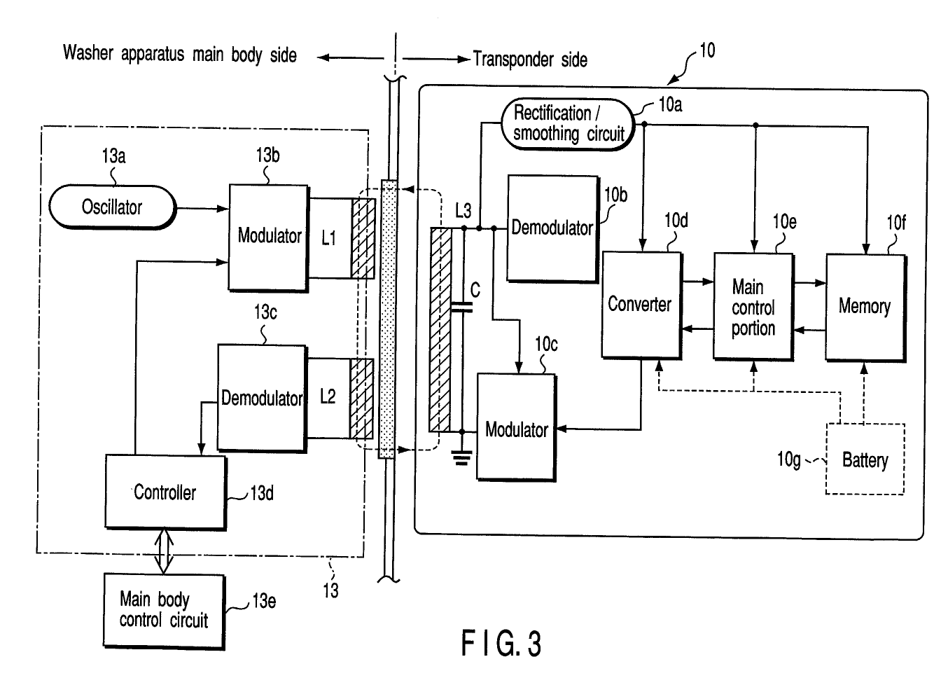

FIG. 3 is a conceptual view of the RFID (transponder). FIGS. 2A and 2B correspond to the RFID (transponder) 10 in FIG. 3, which is generally called a “tag”. Moreover, an RFID transmission/reception circuit main body 13 is provided on the automatic washer disinfector apparatus 1 or a wagon which carries the holder that collectively accommodates therein the cleaning/disinfection objects.

The structure and operation of the RFID (transponder) will now be described with reference to FIG. 3.

In general, there is an RFID (transponder) which is of an “electromagnetic coupling type”, “electrostatic coupling type”, “electromagnetic induction type”, “microwave type” or “optical communication type”. In this embodiment, an “electromagnetic coupling type”, “electromagnetic induction type” and “microwave type” can demonstrate the greater advantages (any other type can also be used).

FIG. 3 uses the “electromagnetic coupling type” as a target, and the RFID transmission/reception circuit main body 13 is connected to a control circuit 13e of the automatic washer disinfector apparatus main body and constituted by a modulation circuit 13b which creates a transmission signal and transmits it to the “tag” of the RFID (transponder) 10, a transmission coil (L1), an oscillator 13a, a reception coil (L2) which receives a signal from the “tag” of the RFID (transponder) 10, a demodulation circuit 13c and a controller 13d which controls the structure.

The “tag” side of the RFID (transponder) 10 is constituted by a coil L3 which transmits/receives a signal, a resonance capacitor C, a demodulation circuit 10b which demodulates a received signal, a converter 10d which reproduces a demodulated signal, a memory 10f which stores data from the converter 10d, and a main control portion 10e which controls the converter 10d and the memory 10f and also controls communication with the main body. In addition, a power supply which drives each circuit in the “tag” is achieved by rectifying/smoothing the signal received by the coil L3 by a rectifying/smoothing circuit 10a and supplying it to each circuit. Reference character 10c denotes a modulation circuit. For further improving the transmission/reception capability, a battery 10g indicated by a dotted line in the drawing may be provided.

The operation will now be described.

In a data write operation, a write command transferred as serial signals from the controller 13d and a radio frequency signal digital-modulated in accordance with write information are applied to the coil Ll. The signal induced in the coil L3 on the “tag” side is amplified and demodulated into the original digital signal by the demodulator 10b. The digitalized serial signal information is converted into a parallel signal by the converter 10d, and the information is stored in the memory 10f.

In an information (data) read operation, a read command supplied from the coil L1 by electromagnetic coupling is detected, and information is read from a specified memory area. Parallel information read from the memory 10f is converted into a serial signal in synchronization with a non-modulation signal supplied from the coil L1, and whether the tank circuit constituted by L3 and C is set in the resonance state is controlled in accordance with that signal in the modulation portion 10c.

On the controller 13d side, information is transmitted/received by detecting whether the tank circuit on the “tag” side vibrates sympathetically through the reception coil L2.

(First Embodiment)

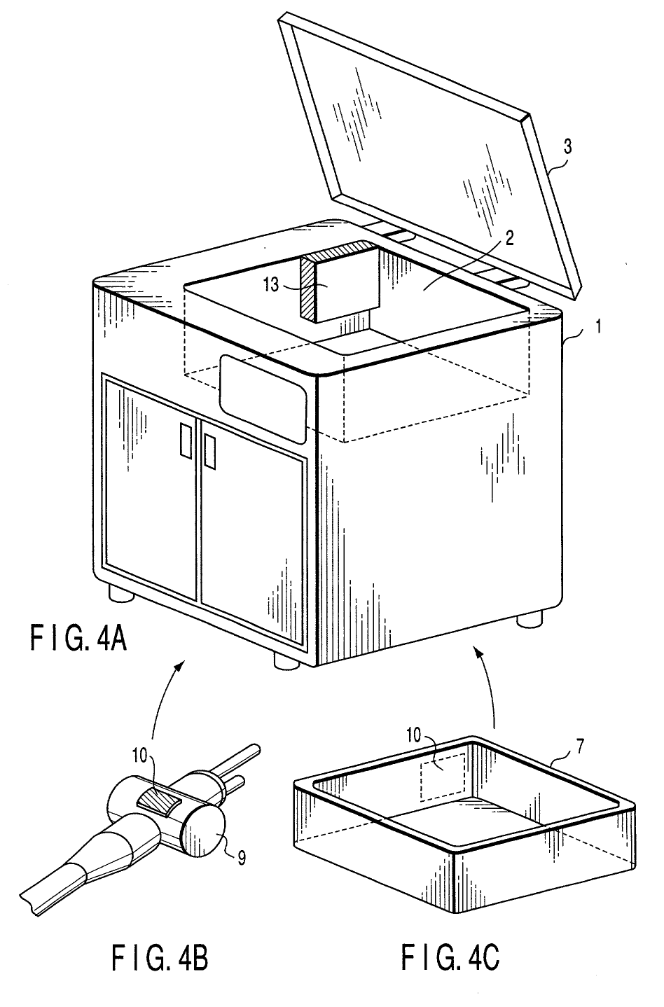

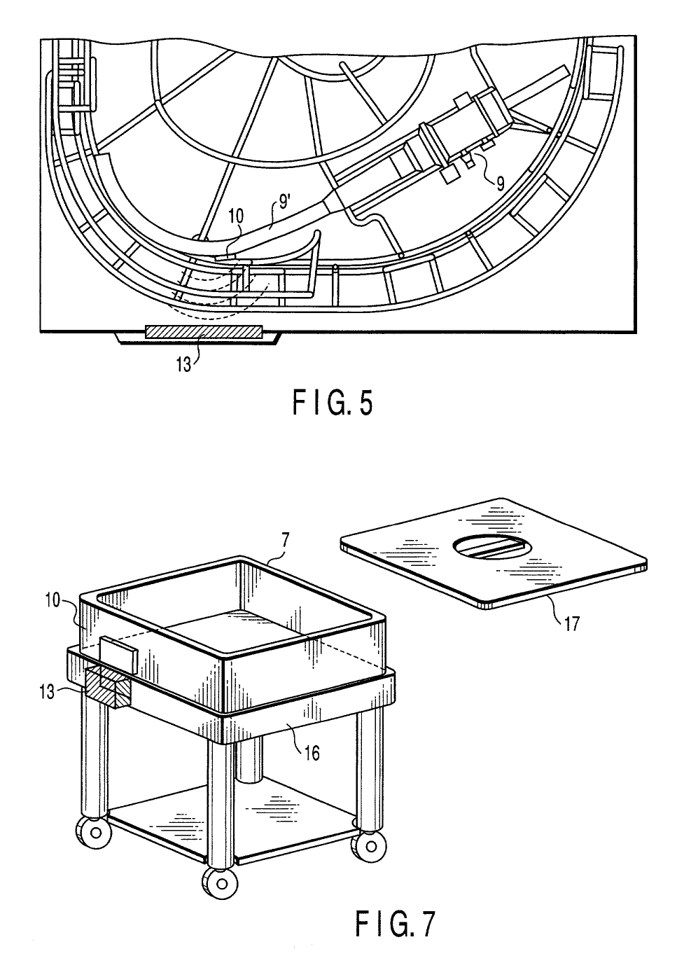

FIGS. 4A, 4B and 4C show a first embodiment. In this embodiment, the “tag” of the RFID is attached to the universal connector 9 of the endoscope as a medical equipment or the holder 7. The “tag” of the RFID can be attached at any position as well as the universal connector 9 of the endoscope and, for example, it may be attached to an universal cord 9′ of the endoscope. FIG. 5 shows that example.

Additionally, in this embodiment, the RFID transmission/reception circuit main body 13 is arranged in a cleaning tank of the automatic washer disinfector apparatus 1. The RFID transmission/reception circuit main body 13 and the “tag” of the RFID attached to the medical equipment operate so as to enter the effect coupling state.

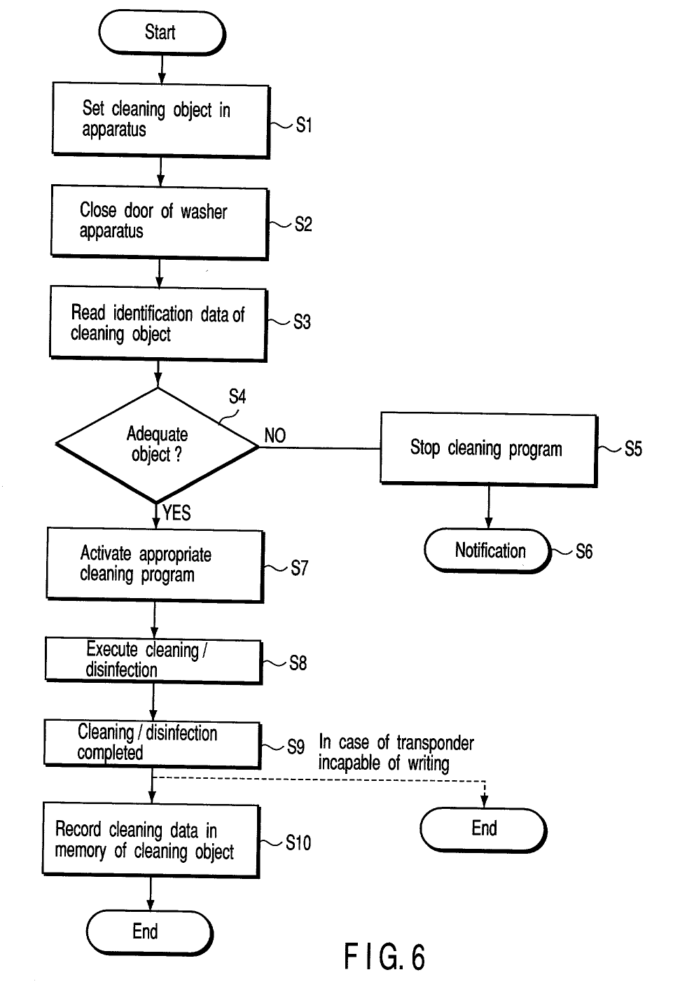

The operation of the first embodiment will now be described hereinafter with reference to the flowchart of FIG. 6.

The cleaning object is first set in the cleaning chamber 2 of the automatic washer disinfector apparatus 1 (step S1), and a door 3 of the washer apparatus is closed (step S2). Upon detecting this operation, a transmission command is supplied from the RFID transmission/reception circuit main body 13 provided to the cleaning chamber 2 to the “tag” provide to the cleaning object. The “tag” returns identification data (for example, a product name, a model name, a lot number, cleaning data and others) of the cleaning object in response to the transmission command.

The RFID transmission/reception circuit main body 13 reads the identification data from the “tag” (step S3), and makes judgment upon whether the cleaning object is an adequate object or whether erroneous settings are made (step S4). If the cleaning object is an adequate object at this point in time, a suitable cleaning/disinfection program is activated (step S7), thereby executing cleaning/disinfection (step S8).

Further, when a series of processing is completed (step S9), the cleaning data (a cleaning time, a disinfection time, data of implementation and others) is transmitted to the “tag” and recorded in a memory in the “tag” as cleaning information of the cleaning object (step S10). This data can be used as reference data when performing next cleaning/disinfection, and so that optimum cleaning/disinfection program can always be executed.

Incidentally, when cleaning parameters are erroneously set by a user, use of the apparatus is prohibited so as not to execute a processing program. Furthermore, by utilizing the RFID (transponder) as a transmission/reception device, data concerning the cleaning object or the cleaning procedure can be easily added to the data of the cleaning object.

Moreover, the apparatus according to this embodiment can be incorporated into a laboratory or hospital as an automatic washer disinfector apparatus of a program control type and used for performing thermal or chemical disinfection and cleaning of medical equipment or apparatuses by a well known method. A set of instruments include apparatuses used for minimally invasive cardiac surgery (MIC) such as an endoscope.

(Second Embodiment)

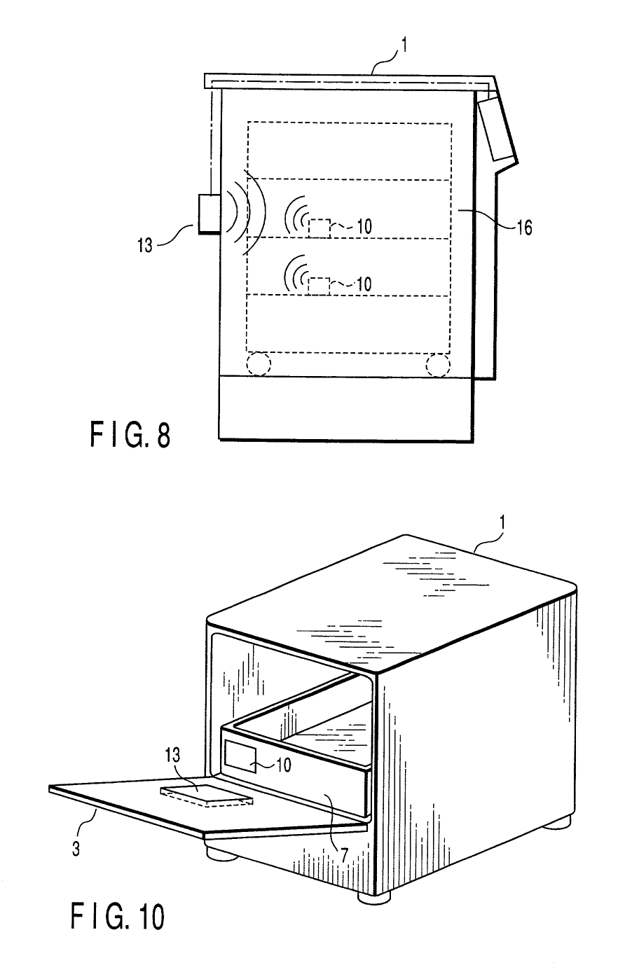

A second embodiment according to the present invention will now be described with reference to FIGS. 7 and 8. in order to adjust the apparatuses for reuse, there is well known type of disinfection/cleaning method with a very high performance, which cleans an apparatus on a patient side by a system compatible with a material of that apparatus in connection with modification of the compatibility with, e.g., optical components of an endoscope. Further, it is possible to use, e.g., an air supply catheter, surgical operation instruments, or a pipette together, in the automatic washer disinfector apparatus.

The apparatuses to be cleaned are accommodated in the holder 7 by a well known method, a holder lid 17 is closed and these apparatuses are distinctively aligned in a cleaning object drawer or a cleaning object wagon 16 to which casters are attached, so that these apparatuses may be processed in the automatic washer apparatus.

In regard to the structure, a data storage medium (=RFID transponder) 10 is attached to the side surface of the holder 7 which accommodates therein the cleaning objects, and an RFID transmission/reception circuit main body 13 is provided on the side surface of the wagon 16 which bears the holder 7 or to the automatic washer disinfector apparatus main body 1 as shown in FIG. 8. The RFID transmission/reception circuit main body 13 is connected to a control circuit of the automatic washer disinfector apparatus and operates similarly as in the first embodiment.

At that moment, selection of a correct program can be set in advance by adequately identifying apparatuses to be processed. The correct cleaning program for all the loaded cleaning objects is selected from the data obtained as a result of transmission/reception, and it is executed.

According to this embodiment, all the medical apparatus or cleaning objects are provided with one dedicated data storage medium or data memory which is firmly coupled with its apparatus and designed so that identification data concerning a type of its cleaning object and any other identification data inherent to the cleaning program are stored or can be added and inputted later. The latter data can be added and supplemented later during processing of the apparatuses, and may include, e.g., data concerning cleaning, an executed procedure and its result, and a time scheduled for storing the cleaning object until the next cleaning of the apparatuses.

The data storage medium (=RFID transponder) 10 is constituted by one active portion (transmitter/receiver) and one passive portion (data memory) in the simplest design. Furthermore, such a data storage medium (=RFID transponder) 10 may be able to read data or write data into the passive portion in some cases.

(Third Embodiment)

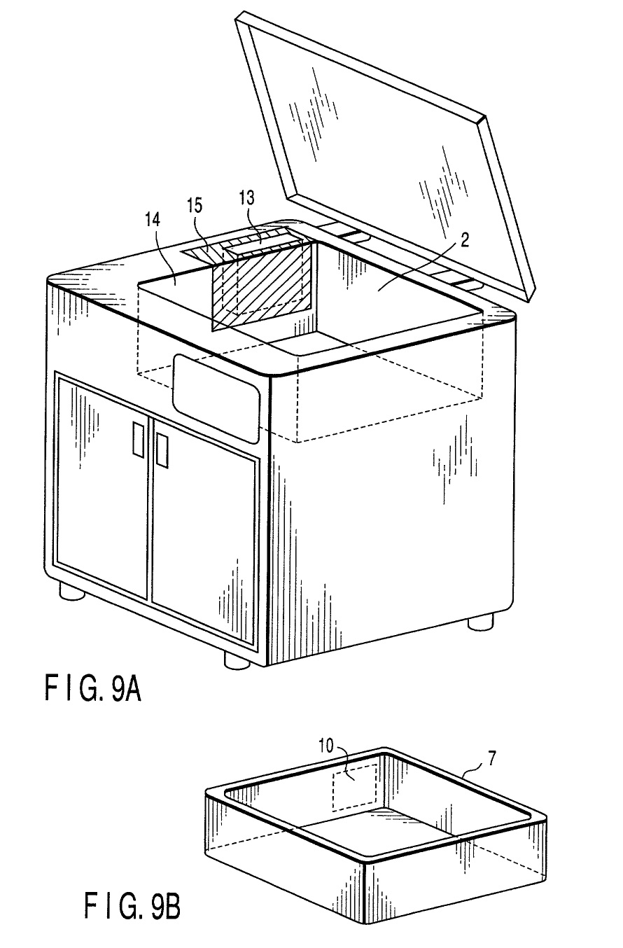

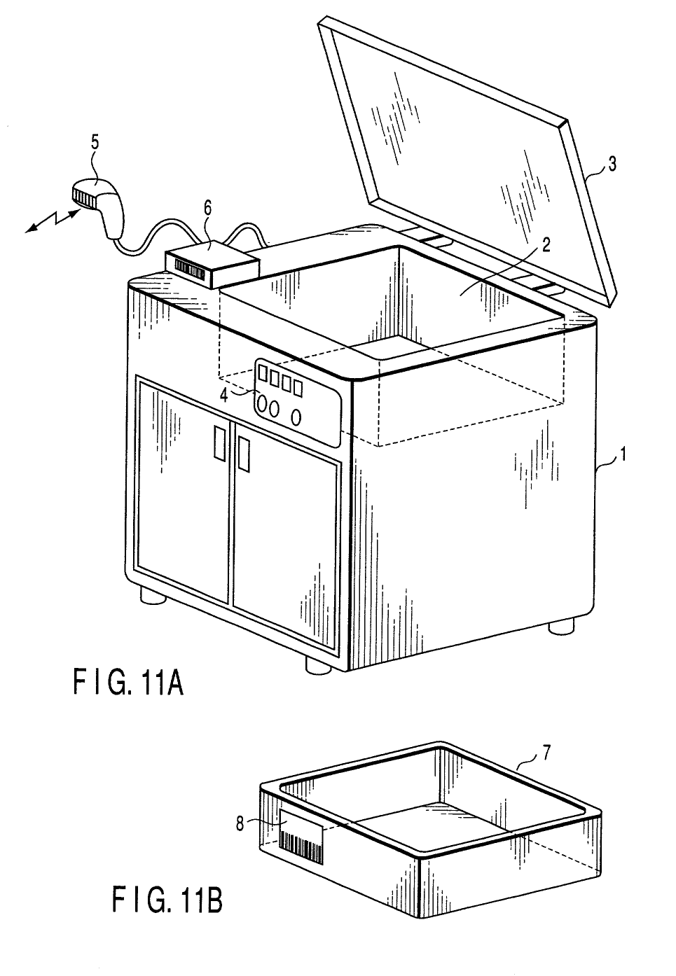

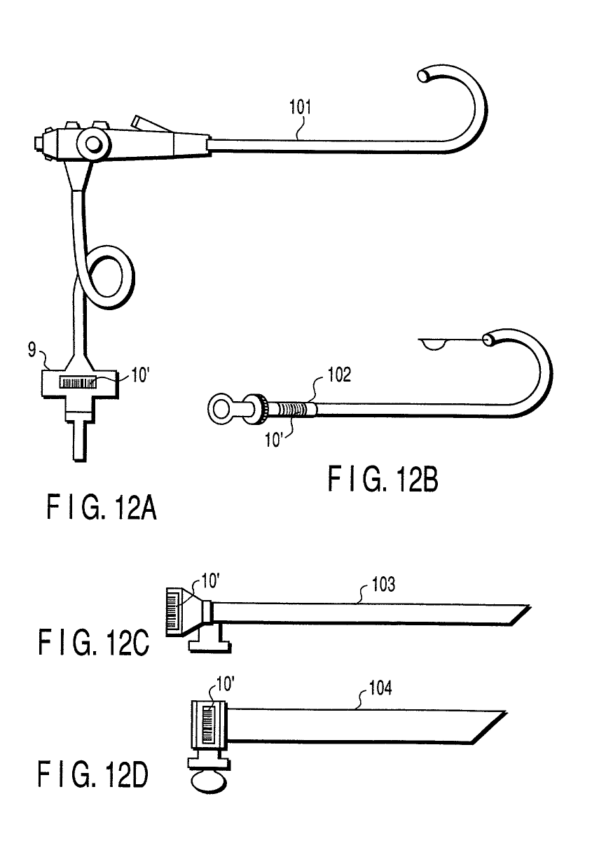

A third embodiment according to the present invention will now be described with reference to FIGS. 9A and 9B.

The RFID transmission/reception circuit main body 13 described in the foregoing embodiments is attached to the outer side of or in the cleaning chamber 2 or, more desirably, it is attached at the rear of one wall of the cleaning chamber 2 closely. In order to enable transmission of data in the optimum state without causing attenuation of the electric field, the walls of the cleaning chamber 2 are formed by a plurality of non-metal foils or (plastic or rubber) plates, and electromagnetic transmission of the identification data of its apparatus read by a non-contact method can further assuredly function.

In this embodiment, in FIG. 9A, a non-metal foil 15 or a plastic or rubber plate is attached in the vicinity of the cleaning tank wall 14 of the cleaning chamber 2, and the RFID transmission/reception circuit main body 13 is provided inside thereof.

Moreover, the data storage medium (=RFID transponder) 10 is provided on the wall surface of the holder 7 shown in FIG. 9B, and attached in the cleaning chamber 2 so as to enter the effect coupling state after putting the cleaning/disinfection objects. When the data storage medium (=RFID transponder) 10 is directly attached to the endoscope or the like, the endoscope or the like may be directly inserted into the cleaning chamber 2.

(Fourth Embodiment)

A fourth embodiment according to the present invention will now be described with reference to FIG. 10.

In the automatic washer disinfector apparatus provided with a mechanism by which the door is opened/closed on the front surface, the RFID transmission/reception circuit main body 13 can be positioned between a metal plate on the inner side of the door 3 and a counterpart on the outer side of the same. Such an assembly method is very convenient for maintenance. When the door of the apparatus is closed, an inquiry is made to the apparatus to be adjusted.

In such a case, one code pattern corresponding to the identification data of that apparatus is generated. When the program starts, the code pattern is tested and recorded by the apparatus. In addition, when using the door which can be folded on the front side of the apparatus (not shown), the common transmission/reception device can be attached so as to facilitate maintenance even if the RFID transmission/reception circuit main body 13 is attached so as to face the front side of the door, for example, it is attached to the lower part of the floor of the cleaning chamber. By adopting this system, even if the coating on the front side of the automatic cleaning apparatus is peeled off, the transponder can be readily controlled or replaced in some cases.

In short, one data storage medium (transponder) is provided for all the cleaning objects, and this data storage medium is designed to be able to read/write data in a non-contact system (read/write device). An inquiry with respect to the loaded cleaning object is made by utilizing the above-described common transmission/reception device after closing and locking the door of the device. An arbitrary number of sets of data of the cleaning objects may be read at arbitrary positions in the cleaning chamber.

As an alternative measure, the cleaning object can be arranged on the fitted wagon, which can possibly perform identification at a fixed position since it is provided with one transponder in some cases. The identification data stored in the data memory of the apparatus is read based on the principle of the electromagnetic transfer. The energy required for data transfer is provided by utilizing energy conduction which is enabled in the form of a plurality of batteries or from the high frequency obtained from the read/write device.

The data inherent in the cleaning object, which is the target of transmission/reception, is stored in the memory, and automatic selection of the necessary cleaning procedure is started.

Incidentally, when cleaning parameters are erroneously set by a user, use of the apparatus is prohibited so as not to execute the processing program. Concurrently with transfer of data, the cleaning data or the like is written in the data storage medium at the end of incorporation of the data inherent to the cleaning object and the procedure which has been executed by using that data in the process of documentation. By the method according to this embodiment, all the cleaning objects can be assuredly identified in accordance with the protocol and actually adjusted for reuse.

According to the foregoing embodiments,

1. in the cleaning chamber of the automatic washer disinfector apparatus having the internally stored programs, medical equipment or apparatuses such as an endoscope or a set of MIC (minimally invasive cardiac surgery) instruments can be identified, an inquiry can be made of the identification data inherent in the cleaning objects of the apparatus, and an optimum program can be activated from some adjustment programs prepared for the apparatuses in the automatic apparatus (optimization of the cleaning/disinfection time);

2. as compared with the conventional bar code method, the capability of detecting the identification data is high (even if conditions of installation of the cleaning objects are erratic, the identification data can be assuredly read/written);

3. as compared with the conventional bar code method, the identification data can be automatically read without performing the operation for reading the identification data each time (reduction in number of steps of a user);

4. by making reference to the identification data, an abnormal cleaning/disinfection step caused due to an erroneous operation can be avoided (prevention of a damage to apparatuses); and

5. by adopting the holder provided with the RFID (transponder), cleaning/disinfection can be efficiently carried out by reading the identification data of a plurality of the cleaning objects in place of performing one-to-one operation with respect to the cleaning objects.

As described above, according to the present invention, there can be provided an automatic washer disinfector apparatus which performs cleaning/disinfection of an object under program control, wherein a data storage medium which stores therein identification data concerning each object is individually provided on each object to be cleaned/disinfected, at least one transmission/reception device which enables data transmission with the data storage medium when an arbitrary object is set in the cleaning chamber of the automatic washer disinfector apparatus is provided on the inside, the surface or the outer side of the automatic washer disinfector apparatus, an appropriate program is selected from a plurality of cleaning/disinfection programs based on the identification data of the object obtained by data communication with the data storage medium, and the selected program is activated, thereby cleaning/disinfecting the object,

therefore, the operation protocol of apparatuses can be easily analyzed by identifying inherent data of a cleaning object and recording data important to the cleaning procedure, and appropriate cleaning/disinfection can be carried out by adequately selecting cleaning conditions of the cleaning object.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An automatic washer disinfector apparatus which cleans/disinfects an object under program control,
wherein a data storage medium which stores therein identification data concerning said each object is individually provided on said each object to be cleaned/disinfected,
wherein at least one transmission/reception device which enables data communication with said data storage medium when an arbitrary object is set in a cleaning chamber of said automatic washer disinfector apparatus is provided on the inside, the surface or the outer side of said automatic washer disinfector apparatus, and
wherein an appropriate program is selected from a plurality of cleaning/disinfection programs based on said identification data of said object obtained by data communication with said data storage medium, and said selected program is activated, thereby performing cleaning/disinfection of said object.

2. The automatic washer disinfector apparatus according to claim 1, wherein said identification data stored in said data storage medium can be read or changed on each data storage medium.

3. The automatic washer disinfector apparatus according to claim 1, wherein said data storage medium is designed as an RFID (transponder).

4. The automatic washer disinfector apparatus according to claim 1, wherein said at least one transmission/reception device is arranged inside or on the surface of an inner wall of said cleaning chamber of said automatic washer disinfector apparatus.

5. The automatic washer disinfector apparatus according to claim 1, wherein said at least one transmission/reception device is arranged on the outer side of said cleaning chamber of said automatic washer disinfector apparatus.

6. The automatic washer disinfector apparatus according to claim 5, wherein said at least one transmission/reception device is closely arranged at the rear of one wall of said cleaning chamber of said automatic washer disinfector apparatus.

7. The automatic washer disinfector apparatus according to claim 1, wherein a wall of said cleaning chamber which is at a part of said at least one transmission/reception device is made of one non-metal foil or plate.

8. The automatic washer disinfector apparatus according to claim 1, wherein said storage medium is further provided on an accommodation device which accommodates therein said object to be cleaned/disinfected.
